(19) United States
(12) Patent Application Publication
(10) Pub. No.: US 2006/0004273 A1
(43) Pub. Date: Jan. 5, 2006

(54) Biological signal sensor on a body surface

(76) Inventor: S. Suave Lobodzinski, Long Beach, CA (US)

Correspondence Address: KNOBBE MARTENS OLSON & BEAR LLP, 2040 MAIN STREET, FOURTEENTH FLOOR, IRVINE, CA, 92614, US

(21) Appl. No.: 11/136,076

(22) Filed: May 24, 2005

Related U.S. Application Data
(60) Provisional application No. 60/573,696, filed on May 24, 2004.

(51) Int. Cl. A61B 5/04

(52) U.S. Cl. 600/395; 600/397

(57) ABSTRACT

An electrically conductive fiber and a coupled biomedical sensor are described. An electrically conductive core of the fiber includes a multiplicity of synthetic conductive filaments and an outer nonconductive fiber layer. The sensor area of the electrically conductive fiber is coated by thin film of silver ink and thin film of silver-silver chloride ink. The electrically conductive fiber's porous surface contacts the thin silver ink coating and the silver coating contacts the silver-chloride ink coating. A surface of the biomedical sensor is coated by a thick film of ionically conductive media, containing an electrolyte, in contact with the silver-chloride coating. The electrolyte diffuses into a porous structure of electrically conductive yarn.

The present application claims priority benefit under 35 U.S.C. § 119(e) from U.S. Provisional Application No. 60/______, filed May 24, 2004, by S. Suave Lobodzinski, titled “A Method and a System for Sensing Biological Signals on the Body Surface,” which is hereby incorporated herein in its entirety by reference.

### BACKGROUND OF THE INVENTION

1. Field of the Invention

This invention relates generally to biomedical sensors, and more particularly, to systems and methods for electrically conductive fibers for biomedical sensors.

2. Description of the Related Art

The practice of medicine utilizes a variety of sensors to measure and record biological signals on the body surface. These include: electrocardiogram, psycho-galvanic reflex of the skin, lactic acid, oxygen concentration, bio-impedance etc. In the electrocardiography applications, the present day ECG recording techniques utilize either non-polarizing Ag/AgCl or polarizing metal electrodes. The electrodes often have either a snap or a wire connector. In some special applications such as catheterization laboratory or magnetic resonance imaging, carbon electrodes and carbon wires are used instead.

Typically, electrodes are one-use products, while lead wires and patient cables are not. Longer-term applications require extensive taping of the electrodes and the connected patient cable to the body, thus making it impractical for longer-term use. Heart monitoring utilizing the ECG electrodes-lead wire-patient cable combinations is used extensively at the coronary and intensive care units in the hospital and in emergency response settings. Typically, the leadwires and patient cables are not sterilized and have been shown to be a source of cross infections in the hospital setting.

Other problems are also commonly encountered in long-term ECG monitoring. Long-term contact of gel sealed under the urethane foam electrode cap causes patient skin irritation and/or skin infection. Half-cell potential variations at the skin-electrode interface due to mechanical deformations of the skin surface under the electrodes cause excessive artifacts in the monitored results. Patient movement that pull on the lead electrode wires cause motion artifacts in the monitored results. Excessive patient perspiration under the electrode cap (gel leakage, baseline wonder etc.) also cause artifacts in the monitored results. There can be EM (electromagnetic) interference signals induced in lead wires.

In addition, current techniques do not provide patient convenience, such as resistance to water (showers or bath) and mechanical exposure (wearing normal clothing and conducting normal daily activities).

### SUMMARY OF THE INVENTION

Embodiments of the present invention address the needs of the long-term, in-hospital and emergency response monitoring markets by providing an inexpensive, one-time use integrated electrode-leadwire-patient cable system that can be either used as a separate device or integrated into a garment for long term ECG monitoring applications.

In an embodiment, a biomedical sensor comprises an electrically conductive fiber, and an ionically conductive medium containing an electrolyte in contact with the electrically conductive fiber. The electrically conductive fiber comprises a multiplicity of flexible, electrically conductive filament cores and two different coatings deposited on the porous outer surface layers of the filaments, where one coating is a thin film having a thickness of about 5 micron of silver ink and where the other coating is a thin film having a thickness about 3 micron silver-silver chloride ink.

The silver ink coating contacts the electrically conductive filament core, and the silver-silver chloride ink coating contacts the ionically conductive media containing electrolyte. The electrolyte of the ionically conductive medium diffuses into the porous filament core of the electrically conductive fiber.

In an embodiment, the silver ink coating comprises nano silver powder with particles having a diameter of about 20 nm to about 30 nm, and a hydrophobic or hydrophilic polymeric binder. In another embodiment, the silver ink coating further comprises optional resins, and an optional cross linking agent.

In an embodiment, the silver-silver chloride ink coating comprises a powder containing approximately 20 nm to approximately 50 nm chlorided silver particles, and a hydrophobic or hydrophilic polymeric binder. In another embodiment, the silver-silver chloride ink further comprises an optional cross linking agent.

At least a part of one end of the electrically conductive fiber forms a lead wire that is not coated with silver ink, silver-silver chloride ink, and the ionically conductive medium.

At least a part of the lengths of the electrically conductive fibers form a lead wire that is covered by a non-electrically conductive layer of yarn and optionally an isolating medium coating in contact with electrically conductive fiber.

At least a part of the other end of the electrically conductive fiber core forms a lead wire termination that is not coated with silver ink, silver-silver chloride ink coatings, and the ionically conductive medium.

In an embodiment, the silver ink coating comprises nano silver powder, silver halide, (hfa)Ag(COD), (hexafluoroacetylacetonato) silver(l) (1,5-cyclo-octadiene), and (hfa)Cu(BTMS), where BTMS is Bis (trimethylsilyl)-acetylene, or combinations thereof.

The hydrophobic polymeric binder has minimal or little water absorbency.

In an embodiment, the flexible, electrically conductive filament core has a thickness from about 0.05 mm to about 0.1 mm. The silver ink coating has a thickness from about 1 micron to about 5 micron. The silver-silver chloride coating has a thickness from about 1 micron to about 5 microns.

In an embodiment, the fiber core filament comprises polyaniline, copper sulfide, silver surface treated polyamide, acrylonitrile, or the like.

In an embodiment, the biomedical sensor further comprises a low porous carbon-containing coating. The low porous carbon-containing coating comprises carbon powder, where the carbon powder comprises graphite powder, carbon black powder, combinations of graphite powder and carbon black powder, or the like.

In another embodiment, the biomedical sensor further comprises a high porous carbon-containing coating. The high porous carbon-containing coating comprises carbon powder, where the carbon powder comprises graphite powder, carbon black powder, combinations of graphite powder and carbon black powder, or the like.

In an embodiment, the content of silver-ink in the silver-ink coating ranges from about 60 weight percent to about 90 weight percent, and the content of the hydrophobic or hydrophilic polymeric binder in the silver-ink coating ranges from about 10 weight percent to about 40 weight percent.

In an embodiment, the content of silver-chloride ink in the silver-chloride coating is less than about 50 weight percent, and the content of the hydrophobic polymeric binder in the silver-chloride ink coating ranges from about 40 weight percent to about 50 weight percent.

In an embodiment, the average adsorbing surface area of the electrically conductive fiber is greater than about 600 m²/g. In an embodiment, the average diameter of the conductive fiber core ranges from about 0.05 mm to about 0.1 mm.

For purposes of summarizing the invention, certain aspects, advantages, and novel features of the invention have been described herein. It is to be understood that not necessarily all such advantages may be achieved in accordance with any particular embodiment of the invention. Thus, the invention may be embodied or carried out in a manner that achieves or optimizes one advantage or group of advantages as taught herein without necessarily achieving other advantages as may be taught or suggested herein.

### BRIEF DESCRIPTION OF THE DRAWINGS

A general architecture that implements the various features of the invention will now be described with reference to the drawings. The drawings and the associated descriptions are provided to illustrate embodiments of the invention and not to limit the scope of the invention.

FIG. 1 is a schematic of an embodiment of a biomedical sensor.

FIG. 2 is a schematic of another embodiment of a biomedical sensor.

FIG. 3 is a cross-sectional view of an embodiment of biomedical sensor.

FIG. 4 is a cross-sectional view of another embodiment of a biomedical sensor.

FIG. 5 illustrates an embodiment of the sensor for very long term wear.

### DETAILED DESCRIPTION OF THE PREFERRED EMBODIMENT

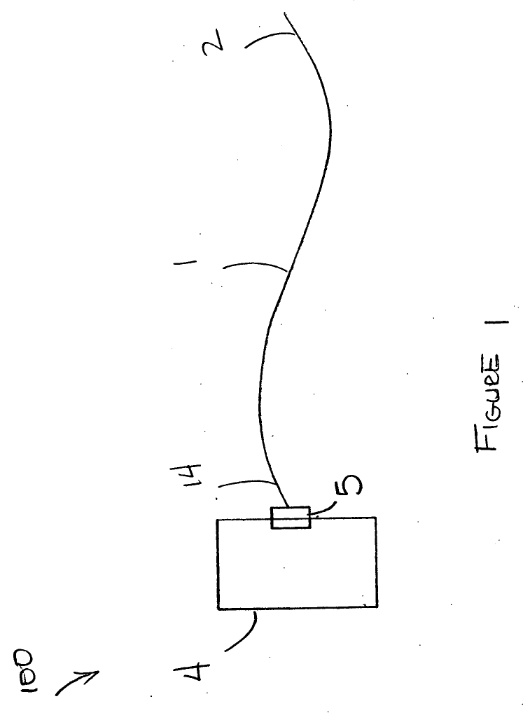

FIG. 1 schematically illustrates an embodiment of a biological signal sensor or biomedical sensor 100. The biomedical sensor 100 comprises a strand of an electrically conductive fiber 1, an ECG amplifier or electrocardiograph 4, and a sensor connector 5 electrically connecting the fiber 1 to the electrocardiograph 4. The biomedical sensor 100 acquires biological signals through contact with a subject and conducts the biological signals to the electrocardiograph 4.

The strand of the electrically conductive fiber 1 forms a sensor tip 2 and a non-sensing end 14. The fiber 1 can be a metal-coated synthetic fiber such as, for example, acrylic, nylon, and the like.

In an embodiment, the sensor connector 5 comprises a nylon frame that encapsulates the non-sensing end of the fiber 14, and protruding domes that squeeze the fiber 14 to a contact thus forming an electrical connection between the fiber and the contact. In an embodiment, the connector comprises a plurality of contacts and the fiber 1 comprises a plurality of fibers.

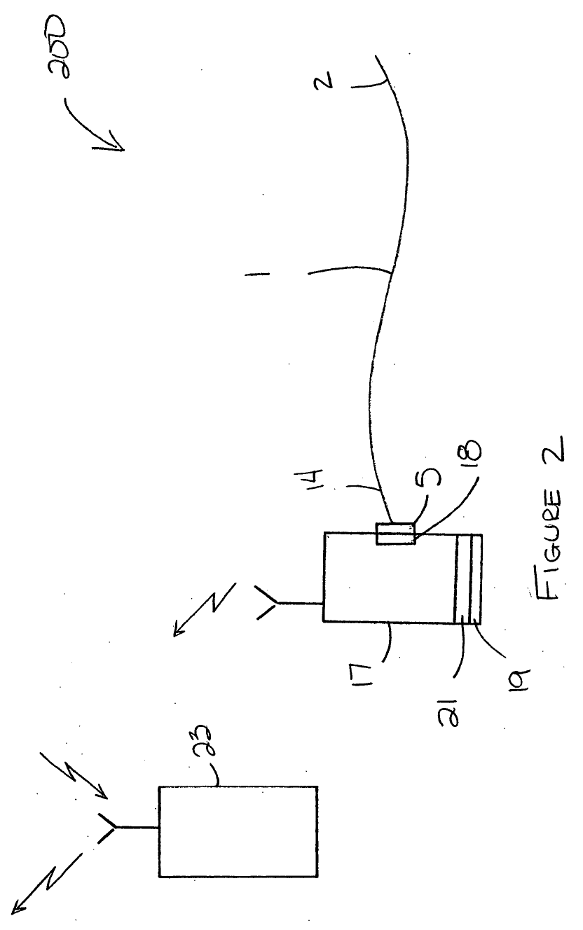

FIG. 2 schematically illustrates another embodiment of a biomedical sensor 200. The biomedical sensor 200 comprises a plurality of sensing fibers 1, each fiber 1 having the sensor tip 2 and the non-sensing end 14, a signal processor/radio transmitter 17, and a network relay device 23. The signal processor/radio transmitter 17 comprises a sensor connector receptacle 18, which electrically connects to the sensor connector 5. The biological sensor 200 acquires biological signals from a subject and conducts the biological signals to the signal processor/radio transmitter 17.

The signal processor/radio transmitter 17 is in communication with the sensing fibers 1 via the connector receptacle 18 and converts the biomedical signals acquired by the sensing fibers 1 into a radio packet for subsequent processing in the wireless network relay device 23. The signal processor/transmitter 17 attaches to the body surface via an adhesive backing layer 21 having an adhesive 19. In an embodiment, the adhesive 19 is a skin neutral kind that does not cause skin irritation or dermatitis. In another embodiment, the adhesive 19 is any medical grade adhesive.

The signal processor/radio transmitter 17 transmits the biological signals to the network relay device 23. In an embodiment, the network relay device 23 is a wearable small computer, such as, but not limited to, a personal digital assistant, an iPaq, and the like, that is equipped with a minimum of two network interfaces. A first network interface communicates with the wearable signal processor/transmitter 17 and the second network interface communicates with local or wide area wireless networks, such as, but not limited to, CDMA, Wi-Fi, Bluetooth, and the like. The network relay device 23 further comprises a microprocessor, network communication software, a user interface, a display, and network interface hardware.

In an embodiment, the network relay device 23 processes the received biological signals and transmits the processed biological signals via the local or wide area wireless networks. In another embodiment, the network relay device 23 transmits the received biological signals via the local or wide area wireless networks for further processing.

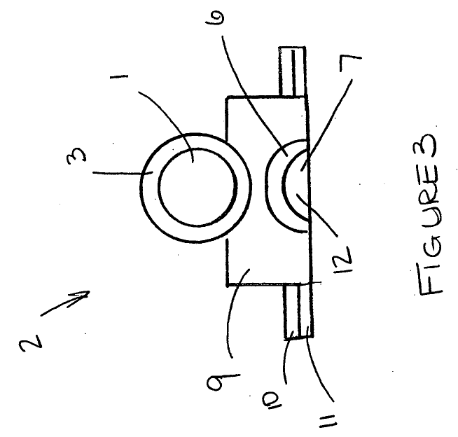

FIG. 3 is a cross-sectional view of an embodiment of the sensor tip 2. The sensor tip comprises the electrically conductive fiber 1, a coating 3, and a sensor-backing layer 9. The coating 3 is adjacent to the fiber 1. In an embodiment, the coating contacts the fiber 1. In a further embodiment, the coating 3 is close to the fiber 1. In an embodiment, the coating 3 is silver-silver chloride ink.

The sensor-backing layer 9 adheres the sensor tip 2 to the subject's skin and promotes the electrical conduction of the biological signals to the sensor 100, 200. The sensor-backing layer comprises outer flanges 10, a sensor reservoir 6, and a cavity 7.

In an embodiment, the sensor-backing layer 9 is made of very thin highly breathable, stretchable, polymeric film. The outer flanges 10 of the film are coated with a non-sensitizing, non-irritating medical grade adhesive 11. In an embodiment, the adhesive 11 is specifically designed for sensitive skin and engineered for longer wear.

The sensor reservoir 6 is made of a spongy material that contains skin permeability enhancers, such as, but not limited to, amino fruit acids, glycolic acid, p-aminobenzoic acid derivatives, salicylic derivatives, triazine derivatives, benzimidazole compounds, bis-benzoazolyl derivatives, methylene bis-(hydroxyphenylbenzotriazole) compounds, 3-imidazol-4-ylacrylic acid, benzene 1,4-di(3-methylidene-10-camphosulfonic) acid, urocanic acid, and the like.

The cavity 7 in the sensor-backing layer 9 can be filled with a conductive paste or gel 12 to promote the acquisition of the biological signals.

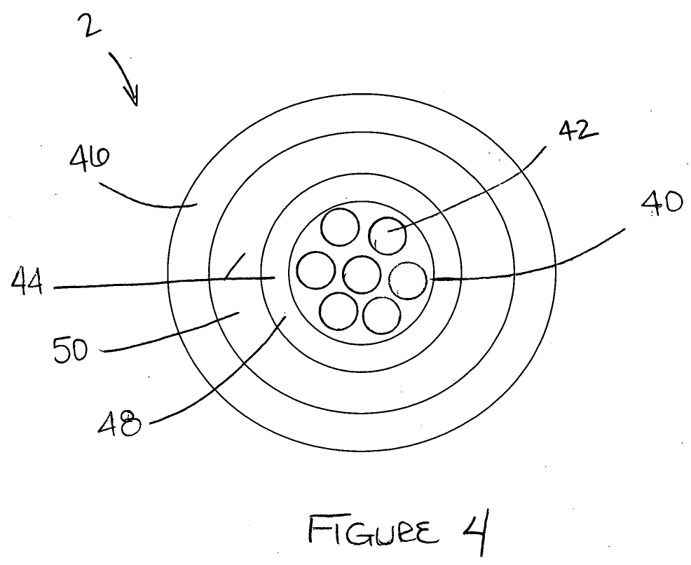

FIG. 4 illustrates a cross-section of another embodiment of the sensor tip 2. The adhesive sensor-backing layer 9 is not shown for clarity. The sensor tip 2 comprises a fiber core 40, which comprises a plurality of electrically conductive filaments 42. Examples of filament material are polyaniline, copper sulfide, silver surface treated polyamide, acrylonitrile, and the like.

In an embodiment, each filament 42 has a porous outer surface coating. The porous coating comprises carbon powder, which comprises at least one of graphite powder and carbon black powder.

In an embodiment, the fiber core 40 has a thickness of approximately 0.05 mm to approximately 0.1 mm, and an average adsorbing surface area of the electrically conductive fiber 1 is greater than approximately 600 m²/g.

The sensor tip 2 further comprises a coating 44 adjacent to the fiber core 1. In an embodiment the coating 44 contacts the fiber core 40. In another embodiment, the coating 44 is close to the fiber core 1. In yet another embodiment, the coating 44 is deposited on the porous outer surfaces of the plurality of filaments 42.

The sensor tip 2 further comprises an ionically conductive medium 46, which comprises an electrolyte. The medium 46 is in electrical communication with the electrically conductive fiber core 40.

In an embodiment, the coating 44 comprises a film comprising silver, where the film has a thickness of about 1 to about 7 microns. In another embodiment, the coating 44 comprises the silver film, where the film has a thickness of about 5 microns. In an embodiment, the coating 44 is silver ink.

In an embodiment, the silver film coating 44 comprises silver powder comprising a plurality of particles having a diameter of between about 20 nm and about 30 nm, and a hydrophobic or hydrophilic polymeric binder in contact with the plurality of particles.

The silver film coating 44 further comprises a resin in or on the silver film coating 44, and a cross-linking agent that cross-links molecules in the coating 44. In an embodiment, the silver content in the silver coating 44 ranges from approximately 60 weight percent to about 90 weight percent, and the binder content in the silver coating 44 ranges from approximately 10 weight percent to approximately 40 weight percent.

In an embodiment, the silver coating 44 comprises at least one of silver powder, silver halide, (hfa)Ag(COD), (hexafluoroacetylacetonato) silver(I) (1,5-cyclo-octadiene), and (hfa)Cu(BTMS).

In an embodiment, the coating 44 is a first coating 48. The sensor tip 2 further comprises a second coating 50. In an embodiment, the second coating 50 comprises a film comprising silver-silver chloride, where the film has a thickness of about 1 to about 5 microns. In another embodiment, the coating 50 comprises the silver-silver chloride film, where the film has a thickness of about 3 microns. In another embodiment, the second coating 50 is silver-silver chloride ink.

In an embodiment, the silver-silver chloride coating 50 comprises chlorided silver powder comprising a plurality of particles having a diameter of approximately 20 nm to approximately 50 nm, and a hydrophobic or hydrophilic polymeric binder in contact with the plurality of particles. In an embodiment, the silver content of the silver-silver chloride in the silver-silver chloride coating 50 is less than approximately 50 weight percent, and the binder content in the silver-silver chloride coating 50 ranges from approximately 40 weight percent to approximately 50 weight percent.

In an embodiment, the silver coating 48 contacts the fiber core 40, and the silver-silver chloride coating 50 contacts the ionically conductive medium 46. The electrolyte contained in the medium 46 diffuses into the fiber core 40 of the electrically conductive fiber 1.

Referring to FIGS. 1, 2, and 4, in an embodiment, at least a part of one end of the electrically conductive fiber 1 forms a lead wire and the lead wire does not contact the coating 44, 48, 50. In another embodiment, at least a part of one end of the electrically conductive fiber core 40 forms a lead wire termination, and the lead wire termination does not contact any of the silver coating 44, 48, the silver-silver chloride coating 50, and the ionically conductive medium 46.

In a further embodiment, at least a part of the electrically conductive fiber core 40 forms a lead wire, and the lead wire is at least partially covered by an electrically nonconductive layer of yarn.

### ECG Sensor Solution as Applied to a Wearable Sensor Shirt

The biomedical sensor is very well suited for signal applications with a specialty garment. The biomedical sensor can also be used in conjunction with a garment that is stitched with the electrically conductive fibers 1 and is in communication with the signal processor/transmitter 17 as shown in FIG. 2, thus forming an integrated sensor-leadwire-patient cable system.

In an embodiment, the biomedical sensor is an ECG sensor. The ECG sensor elements (“electrodes”) are placed on the body surface in preferred ECG locations.

The electric coupling between the garment and the ECG sensor is accomplished through a fastening direct mechanical contact of an electrically conductive hook element mounted on an inner surface of the garment with a loop element of the ECG sensor. The garment's hook element is in electrical communication with an electrically conductive fiber and in electrical communication with a connector.

The ECG sensor mounts on the skin surface. The ECG sensor comprises a conductive gel and a thin layer of silver-silver chloride ink deposited on the backing layer of the ECG sensor. An electrically conductive element connects the silver-silver-chloride surface to the loop element of the ECG sensor. The loop element together with garment's hook element form an electric conduit for conduction of bio currents. The critical skin-electrode interface does not move, thus providing an artifact free ECG signal.

### ECG Sensor Solution as Applied to a Wireless Body Sensor

In an embodiment, biomedical sensors are bonded to the skin directly using pressure-sensitive adhesives. In an embodiment, the biomedical sensors are ECG sensors. In this configuration, the fiber interconnectors link the “electrode” points to a signal processor/transmitter patch. The signal processor/transmitter patch bonds to the skin using a pressure sensitive adhesive layer.

### ECG Sensor Solution as Applied to a Wireless Body Sensor for Very Long Term Wear (Life Long Monitoring)

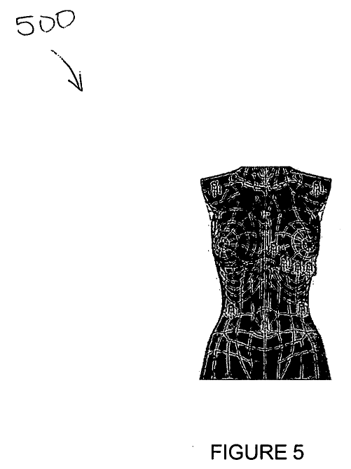

In an embodiment, illustrated in FIG. 5, the biomedical sensor 500 comprises a layer of dermis tattooed with sensor ink. In an embodiment, the biomedical sensor 500 is an ECG sensor and the sensor ink is ECG ink.

The transcutaneously placed ECG sensor ink acts as a sub-dermal “electrode” accumulating ionic charges and converting them into electrons. The stratum corneum (dead layer of the skin) acts as an insulator. A metallized ECG sensor film is applied dry on the body surface. Together, the sub-dermal “electrode” and the ECG Sensor on the skin form a capacitor (insulated dry electrode). The changes of the ECG current in the torso generate an AC signal, which can be measured by a high input impedance differential amplifier. The advantages of this capacitor system as used in conjunction with synthetic interconnections include a very long monitoring period, which not possible with other methods.

While certain embodiments of the inventions have been described, these embodiments have been presented by way of example only, and are not intended to limit the scope of the inventions. Indeed, the novel methods and systems described herein may be embodied in a variety of other forms; furthermore, various omissions, substitutions and changes in the form of the methods and systems described herein may be made without departing from the spirit of the inventions. The accompanying claims and their equivalents are intended to cover such forms or modifications as would fall within the scope and spirit of the inventions.

1. A biomedical sensor, comprising:
an electrically conductive fiber, comprising:
a fiber core, comprising a plurality of electrically conductive filaments, each filament having a porous outer surface; and
a coating adjacent to the fiber core; and
an ionically conductive medium, comprising an electrolyte, the medium being in electrical communication with the electrically conductive fiber;
wherein the coating is deposited on the porous outer surfaces of the plurality of filaments.

2. The biomedical sensor of claim 1, wherein the coating comprises a film comprising silver, the film having a thickness of about 1 to about 7 microns.

3. The biomedical sensor of claim 1, wherein the coating comprises a film comprising silver, the film having a thickness of about 5 microns.

4. The biomedical sensor of claim 2, wherein the coating is a first coating, and further comprising a second coating that comprises a film comprising silver-silver chloride, the film having a thickness of about 1 to about 5 microns.

5. The biomedical sensor of claim 1, wherein the coating is a first coating, and further comprising a second coating that comprises a film comprising silver-silver chloride, the film having a thickness of about 3 microns.

6. The biomedical sensor of claim 4, wherein the silver coating contacts the fiber core, and wherein the silver-silver chloride coating contacts the ionically conductive medium.

7. The biomedical sensor of claim 1, configured such that the electrolyte diffuses into the fiber core of the electrically conductive fiber.

8. The biomedical sensor of claim 2, wherein the silver film coating comprises:
a silver powder comprising a plurality of particles having a diameter of between about 20 nm and about 30 nm; and
a hydrophobic or hydrophilic polymeric binder in contact with the plurality of particles.

9. The biomedical sensor of claim 8, further comprising a resin in or on the silver film coating; and
a cross-linking agent that cross-links molecules in the coating.

10. The biomedical sensor of claim 4, wherein the silver-silver chloride coating comprises:
a chlorided silver powder comprising a plurality of particles having a diameter of approximately 20 nm to approximately 50 nm; and
a hydrophobic or hydrophilic polymeric binder in contact with the plurality of particles.

11. The biomedical sensor of claim 1, wherein at least a part of one end of the electrically conductive fiber forms a lead wire, and wherein the lead wire is not contacting the coating.

12. The biomedical sensor of claim 1, wherein at least a part of the electrically conductive fiber core forms a lead wire, wherein the lead wire is at least partially covered by an electrically nonconductive layer of yarn.

13. The biomedical sensor of claim 4, wherein at least a part of one end of the electrically conductive fiber core forms a lead wire termination, wherein the lead wire termination is not contacted by any of the silver coating, the silver-silver chloride coating, and the ionically conductive medium.

14. The biomedical sensor of claim 2, wherein the silver coating comprises at least one of silver powder, silver halide, (hfa)Ag(COD), (hexafluoroacetylacetonato) silver(I) (1,5-cyclo-octadiene), and (hfa)Cu(BTMS).

15. The biomedical sensor of claim 4, wherein the fiber core has a thickness of approximately 0.05 mm to approximately 0.1 mm, wherein the silver coating has a thickness of approximately 1 micron to approximately 5 microns, and wherein the silver-silver chloride coating has a thickness of approximately 1 micron to approximately 5 microns.

16. The biomedical sensor of claim 1, wherein the plurality of filaments comprises a material selected from the group consisting of polyaniline, copper sulfide, silver surface treated polyamide, and acrylonitrile.

17. The biomedical sensor of claim 1, wherein the coating comprises carbon powder comprising at least one of graphite powder and carbon black powder.

18. The biomedical sensor of claim 2, wherein a content of silver in the silver coating ranges from approximately 60 weight percent to about 90 weight percent.

19. The biomedical sensor of claim 8, wherein a content of the hydrophobic or hydrophilic polymeric binder in the silver film coating ranges from approximately 10 weight percent to approximately 40 weight percent.

20. The biomedical sensor of claim 10, wherein a content of the silver-silver chloride in the silver-silver chloride coating is less than approximately 50 weight percent, and wherein a content of the hydrophobic polymeric binder in the silver-silver chloride coating ranges from approximately 40 weight percent to approximately 50 weight percent.

21. The biomedical sensor of claim 1, wherein an average adsorbing surface area of the electrically conductive fiber is greater than approximately 600 m²/g, and wherein a diameter of the conductive fiber core ranges from approximately 0.05 mm to approximately 0.1 mm.
